(19) United States
(12) Patent Application Publication
(10) Pub. No.: US 2017/0151351 A1
(43) Pub. Date: Jun. 1, 2017

(54) Gd-ENCAPSULATED CARBON DOTS AND METHODS OF MAKING AND USING THEREOF

(71) Applicant: University of Georgia Research Foundation, Inc., Athens, GA (US)

(72) Inventors: Jin Xie, Athens, GA (US); Hongmin Chen, Athens, GA (US); Zibo Li, Chapel Hill, NC (US)

(21) Appl. No.: 15/361,784

(22) Filed: Nov. 28, 2016

Related U.S. Application Data
(60) Provisional application No. 62/260,900, filed on Nov. 30, 2015.
(60) Provisional application No. 62/260,525, filed on Nov. 28, 2015.

(51) Int. Cl. A61K 49/18; C01B 31/02

(52) U.S. Cl. CPC A61K 49/183; A61K 49/1866; C01B 31/02

(57) ABSTRACT

Gd-encapsulated carbonaceous dots (Gd@C-dots) hold great potential in clinical translation as Ti contrast agent for magnetic resonance imaging. However, current synthetic techniques yield particles with poor size control; hence, time-consuming size selection is often needed to obtain particles of desired sizes. Disclosed is a process whereby mesoporous silica nanoparticles are used as templates for size-controlled synthesis of Gd@C-dots. The disclosed methods involve calcining a mixture comprising a mesoporous silica nanoparticle, a gadolinium-containing compound, and a chelator, thereby forming the nanoparticles of gadolinium within the mesoporous silica nanoparticle; and removing the mesoporous silica nanoparticle from the nanoparticles of gadolinium.

### CROSS REFERENCE TO RELATED APPLICATIONS

This application claims the benefit of priority to U.S. Provisional Application 62/260,525, filed Nov. 28, 2015, and U.S. Provisional Application 62/260,900, filed Nov. 30, 2015, both of which are incorporated by reference herein in their entireties.

### ACKNOWLEDGMENT OF GOVERNMENT SUPPORT

This invention was made with government support under Grant No. CA140666 awarded by the Department of Defense. The government has certain rights in this invention.

### FIELD

The subject matter disclosed herein generally relates to nanoparticles containing a gadolinium encapsulated in a carbonaceous shell, and methods of making and using thereof.

### BACKGROUND

MRI is one of the most widely used diagnostic tools in the clinic. MRI features a number of advantages such as noninvasive, high spatial and temporal resolutions, as well as good soft tissue sensitivity (Na, H. et al., Adv. Mater. 2009, 21:2133; Chen, H., Mater. Sci. Eng. R. 2013, 74:35). Despite this, the intrinsic signal different among tissues, especially that between diseased and normal tissues, is often suboptimal. Hence, a MRI contrast agent is often injected during or prior to scanning to improve imaging quality. So far, the most commonly used MRI contrast agents are based on Gd(III) because it affords seven unpaired electron spins, which leads to high T₁ contrast. However, free Gd(III) is highly toxic (Ersoy, H. et al., J. Magn. Reson. Imaging 2007, 26:1190). To suppress the toxicity, a multi-dentate ligand is used to complex with Gd(III) to suppress its toxicity while maintaining the T₁ contrast ability. Examples include Gd-DTPA, gadoteric acid, Gd-DO3A-butrol, and gadodiamide, etc. (Zhou, Z., et al., Wiley Interdiscip. Rev. Nanomed. Nanobiotech. 2013, 5:1). But recent studies suggest that despite strong chelation, Gd may be released from the complex in vivo. This poses toxicities to the host, especially to patients with compromised renal functions, in which case a rare disease called nephrogenic systemic fibrosis (NSF) may be induced (Lee, S., et al., Wiley Interdiscip. Rev. Nanomed. Nanobiotech. 2014, 6:196; Meloni, A., et al., Haematologica 2009, 94:1625; Caravan, P., et al., Chem. Rev. 1999, 99:2293; Chrysochou, C., et al., Clin. J. Am. So.c Nephro. 2010, 5:484). Due to this reason, there is an urgent need to find alternative Gd agents with minimized Gd leakage while affording comparable or enhanced contrast abilities (Terreno, E., et al., Chem. Rev. 2010, 110:3019).

One approach that has been extensively exploited is doping Gd into a nanoparticle capsule so that the release of Gd(III) can be curtailed. Examples along this direction include Gd₂O₃ nanoparticles (Bridot, J., et al., J. Am. Chem. Soc. 2007, 129:5076), Gd-loaded silica nanoparticles (Vivero-Escoto, J., et al., Small 2013, 9:3523), Gd-doped Fe₃O₄ nanoparticles (Zhou, Z., et al., Adv. Mater. 2012, 24:6223), and Gd-coordinated polymers (Lim, C., et al., Biomaterials 2013, 34, 6846; Yang, H., et al., Adv. Funct. Mater. 2014, 24:738). However, due to their size, these nanoparticles are largely trapped in reticuloendothelial system (RES) organs, such as the liver, spleen, and bone marrow (Sancey, L., et al., ACS Nano 2015, 9:2477). In these organs, the nanocapsules will eventually degrade, which poses risks of long-term toxicities to the host (Id.).

In another approach, Gd-encapsulated carbon dots (Gd@C-dots) were developed as a new type of T₁ contrast agent (Chen, H., et al., Adv. Mater. 2014, 26:6761). Gd@C-dots exhibit high r₁ relaxivity (at least twice that of Gd-DTPA) (Kim, T., et al., J. Am. Chem. Soc. 2011, 133:2955; Kalavagunta, C., et al., Contrast media Mol. Imaging 2014, 9:169), strong photoluminescence, and low toxicity. More uniquely, carbon is not a biodegradable material. Studies showed that Gd@C-dots remained intact in harsh biological environments (Chen, H., et al., Adv. Mater. 2014, 26:6761), leading to low toxicities both in vitro and in vivo. These properties suggest great potential of Gd@C-dots as a safe alternative to Gd complexes.

One problem, however, is the relatively low production yield of the Gd@C-dots. In previous studies, Gd@C-dots were made by directly calcinating of Gd-DTPA in the air. The raw products contain a broad spectrum of carbon species (from 2 to 1000 nm). To obtain particles of desired sizes, multiple rounds of purifications and size-enrichments are needed, which is time-consuming (Luo, P., et al., J. Mater. Chem. B 2013, 1:2116; Wang, Y., et al., J. Mater. Chem. C 2014, 2:6921). Meanwhile, size is expected to have a major impact on the magnetic and optical properties of Gd@C-dots, and in turn affect their performances as imaging probes. A synthetic approach that allows for good size control is therefore valuable for both fundamental studies and clinical applications of the Gd@C-dots. The subject matter disclosed herein addresses these and other needs.

### SUMMARY

In accordance with the purposes of the described materials, compounds, compositions, articles, and methods, as embodied and broadly described herein, the subject matter described herein, in one aspect, relates to compositions and methods for preparing and using such compositions. In a further aspect, the disclosed subject matter relates to methods of preparing a nanoparticle comprising a metal encapsulated in an amorphous carbon shell. In particular examples, the disclosed subject matter relates to a nanoparticle comprising gadolinium encapsulated in an amorphous carbon shell and methods of preparing such nanoparticles. In the disclosed methods a mixture comprising a mesoporous silica nanoparticle, a gadolinium-containing compound, and a chelator is calcined, thereby forming the nanoparticles of gadolinium within the mesoporous silica nanoparticle; and the mesoporous silica nanoparticles can be removed from the nanoparticles of gadolinium (or other metal). Methods of functionalizing the disclosed nanoparticles are also disclosed. Methods of using the disclosed nanoparticles, e.g., for imaging are also disclosed.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

The accompanying figures, which are incorporated in and constitute a part of this specification, illustrate several aspects of the disclosure, and together with the description, serve to explain the principles of the disclosure.

FIG. 1A contains a schematic illustration showing the procedure of Gd@C-dots preparation. FIG. 1B contains TEM images of MSN-3, MSN-7, and MSN-11. FIG. 1C shows the FT-IR spectra of MSN-3, MSN-7, and MSN-11. FIG. 1D shows the TGA spectrum of MSN-3.

FIG. 2A shows the FT-IR spectrum of MSN-3 after calcination. FIG. 2B shows the FT-IR spectra of Gd@C-dots made from MSN-3, -7, and -11, respectively. FIG. 2C contains TEM images of Gd@C-dots made from MSN-3, -7, and -11, respectively. FIG. 2D shows the statistic size distributions of Gd@C-dots. Results are based on TEM images (>100 particles). FIG. 2E shows an EDX analysis of Gd@C-dots. FIG. 2F shows the TGA spectrum of 3.0 nm Gd@C-dots.

FIG. 3A shows the absorbance of 3.0, 7.4, and 9.6 nm Gd@C-dots. FIG. 3B shows the photoluminescent spectra of 3.0, 7.4, and 9.6 nm Gd@C-dots under excitation of different wavelengths. FIG. 3C contains photographs of Gd@C-dot solutions under natural (top) and UV light (bottom, 365 nm). FIG. 3D shows the photo-stability of fluorescein, CdSe/ZnS QDs, and Gd@C-dots. All samples were continuously irradiated by a 120 W xenon lamp.

FIG. 4A contains T₁-weighted MR images of Gd@C-dot samples in 1% agarose. FIG. 4B shows the r₁ relaxivity assessment, which is based on results from FIG. 4A.

FIGS. 5A-5D contain data from stability and cytotoxicity studies. FIGS. 5A shows the photoluminescence of Gd@C-dots at different pH. FIGS. 5B shows the Gd release from Gd@C-dots at different time points. FIGS. 5C shows cell viability results. 25 mM Ca(II) was added to the incubation medium. Gd-DTPA was studied as a comparison. No cell morphology change was observed when 3.0 nm Gd@C-dots were incubated with cells (FIGS. 5D).

FIG. 6 shows in vitro cancer cell targeting. Microscopic images of U87MG cells after incubating with RGD-Gd@C and Gd@C-dots for 1 h. Scale bar: 100 μm.

FIG. 7A contains T₁-weighted coronal MR images. Gd@C-dots or RGD-Gd@C-dots (0.1 mmol Gd/kg) were intravenously injected into U87MG tumor bearing mice. Images were acquired at 0, 0.5, 1, 2 and 4 h. Significant signal enhancement was observed in tumors of animals injected with RGD-Gd@C-dots. Meantime, Gd@C-dots induced little signal enhancement in tumors. FIG. 7B shows the relative signal change at different time points, based on the imaging results from FIG. 7A. FIG. 7C contains T₁-weighted transverse MR images. For both types of nanoparticles, strong signals in the bladder were observed quickly after the particle injection, indicating fast and efficient renal clearance. FIG. 7D shows signal changes in the bladder, liver, and kidney, based on region of interest (ROI) analysis on the images FIG. 7C.

FIG. 8A shows Zeta potentials of MSN-3, MSN-7, and MSN-11. FIG. 8B shows Zeta potential of calcined MSN-3.

FIG. 9 shows the Zeta potential of Gd@C-dots.

FIGS. 10A-10F show XPS spectra of Gd@C-dots. FIG. 10A shows a full XPS, FIG. 10B shows C₁ₛ, FIG. 10C shows N₁ₛ, FIG. 10D shows O₁ₛ, FIG. 10E shows Gd_{4d}, and FIG. 10F shows Gd_{3d} of 3.0 nm Gd@C-dots.

FIG. 11 shows the R₂ relaxivities of Gd@C-dots.

FIG. 12 is a group of photographs showing the Colloidal stability of Gd@C-dots and RGD-Gd@C-dots. Gd@C-dots and RGD-Gd@C-dots remained stable for over 6 months in aqueous solutions and there was no visible precipitation.

FIG. 13 shows an immunofluorescence imaging study with tumor sections taken from animals injected with RGD-Gd@C-dots and Gd@C-dots. Strong fluorescence from Gd@C-dots was observed from the RGD-Gd@C-dot group but not the Gd@C-dot group. Scale bars, 100 μm.

FIG. 14A shows the photoluminescence analysis of urine samples (after purification) taken 120 min from mice injected with RGD-Gd@C-dots. FIG. 14B contains a TEM analysis of urine samples from mice injected with RGD-Gd@C-dots (scale bar: 10 nm).

### DETAILED DESCRIPTION

The present disclosure can be understood more readily by reference to the following detailed description and the Examples included therein and to the Figures and their previous and following description.

As used in the specification and the appended claims, the singular forms “a,” “an,” and “the” include plural referents unless the context clearly dictates otherwise. Thus, for example, reference to “a particle” includes mixtures of two or more such particles, reference to “the compound” includes mixtures of two or more such compositions, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. Unless stated to the contrary “about” a particular value means within 5% of the particular value, e.g., within 2% or 1% of the particular value.

By “amorphous” is meant non-crystalline and without structural order over a long range, e.g., a majority of the nanoparticle. An amorphous shell can contain some ordered structure over a short range atomic length scale, but the majority of the shell is not ordered and non-crystalline.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

### Methods of Making

C-dots hold great potential in a wide range of applications such as in vivo imaging, intraoperative imaging, and immunofluorescence staining (Lim, S., et al., Chem. Soc. Rev. 2015, 44:362). A myriad of C-dot synthesis methods have been developed, including laser ablation, electrochemical oxidation, chemical oxidation, thermal carbonization, pyrolysis, and microwave irradiation (Wang, Y., et al., J. Mater. Chem. C 2014, 2:6921). Despite the diversity in synthesis strategies, however, it is generally challenging to control the size of C-dots. This is because the raw products from conventional syntheses often contain carbon species of varied sizes and architectures (Id.). Multiple rounds of washing and purification are often required to obtain particles of the desired qualities, leading to time-consuming preparation and low production yields (Miao, P., et al., Nanoscale 2015, 7:1586). Moreover, as-synthesized C-dots often show weak luminescence, and require a post-synthesis surface modification to “enlighten” the particles (Yang, S., et al., J. Am. Chem. Soc. 2009, 131:11308). The surface modification adds to the complications of quality control (Sun, Y., et al., J. Am. Chem. Soc. 2006, 128:7756).

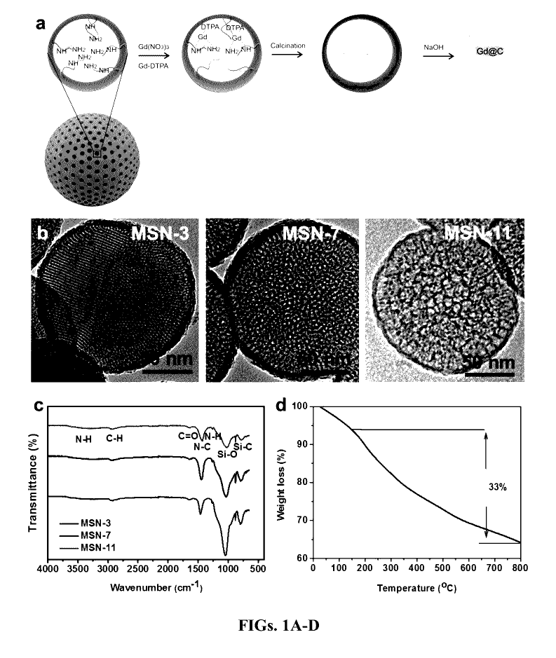

Disclosed herein is a MSN-templated synthesis method for the preparation of gadolinium carbon dots. MSNs of different pore sizes were prepared using cetyl trimethylammonium bromide (CTAB)-templated co-condensation method, followed by alkaline-etching (Chen, Y., et al., Small 2011, 7:2935). The MSNs can be used as reactors, into which Gd precursors are loaded. The resulting conjugates can then be calcined, leading to formation of Gd@C-dots throughout the silica matrix (FIG. 1A). Restrained by the dimension of the pores, however, the nanoparticle growth is limited, yielding homogeneous products whose sizes mold by the silica pores. Taking MSN-3, -7, and -11 for instance (3, 8, 10 indicate the average pore size of the MSNs), the resulting Gd@C-dots are on average 3.0, 7.4, and 9.6 nm in size, respectively. The resulting nanoparticles show excellent magnetic and optical properties. In particular, 3.0 nm Gd@C-dots possess very high relaxivity of 10 mM⁻¹s⁻¹ and quantum yield of 30.2%. When coupled with a tumor targeting ligand, c(RGDyK), the resulting conjugates show great tumor targeting, with the unbound particles quickly cleared from the host. The synthetic method addresses one critical problem in synthesis of carbon nanoparticle, promising clinical translation of Gd@C-dots as a new type of imaging agents. The disclosed method permits preparation of homogenous particles without any size enrichment steps. More surprisingly, the resulting Gd@C-dots show excellent luminescence properties without further surface modification.

Including organofunctional silanes like APS in the MSN preparation has proven to be helpful for Gd@C-dot generation: when using MSNs made from pure TEOS as reactors, the same calcination protocol failed to produce Gd@C-dots. It is postulated that by using aminosilanes in coagulation, many primary and secondary amine groups are introduced into the silica matrix. These amines may loosely complex with Gd precursors that are loaded into the MSNs. More importantly, the amines serve as de facto defects in the silica matrix, which are oxidized during calcination. The oxidization is likely facilitated by metallic centers, causing carbonization and the growth of a carbon shell surrounding Gd. The growth, however, is limited by the volume of the silica cavity, leading to formation of Gd@C-dots of similar sizes.

The size effect on the r₁ relaxivity of Gd@C-dots is to some degree unexpected. For conventional Gd complexes or nanoparticles, increasing the size of the agents would result in an increased rotation time, leading to r₁ increase (Wang, Y., et al., Adv. Mater. 2015, 27:3841). For Gd@C-dots, however, size is inversely correlated to r₁. In fact, it is counterintuitive that Gd encapsulated carbon species have a high r₁. According to classic models, direct Gd-water interaction is needed for T₁ shortening, which, in our case, is not happening when Gd is encased within a layer of carbon. Yet, enhanced r₁ relaxivities are observed in different types of Gd encapsulated carbon species, including not only the disclosed Gd@C-dots, but also Gd loaded carbon tubes and fullerene (Holt, B., et al., ACS Appl. Mater. Interfaces, 2015, 7:14593). A possible explanation was given by Wilson, who proposed that the encased Gd cations may affect the electron density of the carbon shell, thus enabling the relaxation of water molecules to occur at the carbon shell without direct Gd-water interaction (Shu, C., et al., Biocon. Chem. 2009, 20:1186). It is also possible that the large number of hydroxide and carbonyl groups on the carbon shell play a role by facilitating the proton exchange of protons with the surroundings (Bolskar, R., et al., J. Am. Chem. Soc. 2003, 125:5471; Laus, S., et al., J. Phys. Chem. C 2007, 111:5633; Sitharaman, B., et al., Chem. Commun. 2005, 3915; d) Sithararnan, B., et al., Int. J. Nanomed. 2006, 1:291). With a reduced particle size, the ratio of surface or near-surface Gd contributing to the T₁ shortening is increased, thus leading to an enhanced r₁.

The luminescence of C-dots is also an interesting phenomenon and the mechanism has been a subject of debate. However, it is increasingly accepted that the radiative recombinations of the surface-confined electrons and holes are responsible for the luminescence (Zhu, S., et al., Adv. Funct. Mater. 2012, 22:4732). Previously, the most intensively studied factor for luminescence was surface passivation (Luo, P., et al., J. Mater. Chem. B 2013, 1:2116). It was found that proper post-synthesis surface modification, such as oxidation or tethering molecules to the particles surface, could dramatically enhance the luminescence intensity of C-dots (Liu, F., et al., Adv. Mater. 2013, 25:3657). In fact, post-synthesis surface modification is often times an essential step in preparation of luminescent C-dots (Yang, S., et al., J. Am. Chem. Soc. 2009, 131:11308). In the disclosed methods, however, no post-synthesis treatment is applied; yet, QY as high as 30.2% was observed. The very high QY could be attributed to a reduced particle size that causes more defects on the surface carbon, leading to more energy states to trap excitons during excitations (Pan, D., et al., Adv. Mater. 2010, 22, 734; Lingam, K., et al., Adv. Funct. Mater. 2013, 23:5062). It may also be due to addition of a non-carbon dopant, which is found recently as a cause of increased QY of C-dots (Sun, Y., et al., J. Phys. Chem. C 2008, 112, 18295; Tian, L., et al., Chem. Mater. 2009, 21:2803; Dong, Y., et al., Angew. Chem. Int. Ed. 2013, 52:7800).

Disclosed herein are MSN-templated synthetic methods to prepare Gd@C-dots. With the disclosed methods, different sizes of Gd@C-dots can be prepared in one-step, avoiding time-consuming purification that is required in conventional methods. In certain examples, 3.0 nm Gd@C-dots showed zero Gd leakage in physiological conditions, low toxicity, strong luminescence, high r1 relaxivity, and efficient renal clearance, suggesting their great potential as a novel type of T1 contrast agent. The disclosed methods can be extended to prepare other types of metal doped C-dots with good size and property control.

Specifically, disclosed herein is a method of forming nanoparticles of gadolinium encapsulated in an amorphous carbon shell, comprising calcining a mixture comprising a mesoporous silica nanoparticle, a gadolinium-containing compound, and a chelator, thereby forming the nanoparticles of gadolinium within the mesoporous silica nanoparticle; and removing the mesoporous silica nanoparticle from the nanoparticles of gadolinium. The chelators can form complexes with gadolinium and other metals. By calcining the metal-chelate complexes, a nanoparticle where the metal is encapsulated in an amorphous carbon shell can be produced. Calcining can be performed at from about 150° C. to about 300° C. in a muffle furnace or similar furnace. In specific examples, calcining can be performed at about 200° C.

Removing the mesoporous silica nanoparticle can be accomplished by dissolving it in a base and isolating the nanoparticles of gadolinium. Bases such as sodium hydroxide, potassium hydroxide, lithium hydroxide, calcium hydroxide, sodium carbonate, potassium carbonate, ammonium hydroxide, and the like can be used.

The mesoporous silica nanoparticle can be prepared by contacting a tetraalkyl orthosilicate and organofunctional silane in the presence of a tetraalkylammonium halide. Examples of suitable tetraalkyl orthosilicates are tetraethylorthosilicate, tetramethylorthosilicate, tetrapropylorthosilicate, and the like. Examples of organofunctional silanes aminosilanes include [3-(2-Aminoethylamino)propyl]trimethoxysilane, and the like. Examples of tetraalkylammonium halide include cetyltrimethylammonium bromide, cetyltrimethylammonium chloride, benzalkonium bromide, benzalkonium chloride, dodecyldimethyl ammonium bromide, dodecyldimethylammonium chloride, and other quaternary ammonium halides.

The mesoporous silica nanoparticle can have an average diameter of from about 100 nm to about 200 nm. For example, the mesoporous silica nanoparticles can have an average diameter of from about 110 nm to about 200 nm, from about 120 nm to about 200 nm, from about 130 nm to about 200 nm, from about 140 nm to about 200 nm, from about 150 nm to about 200 nm, from about 160 nm to about 200 nm, from about 100 to about 180 nm, from about 110 to about 160 nm, from about 120 nm to about 160 nm, or from about 130 nm to about 150 nm. In a specific example, the mesoporous silica nanoparticle can have an average diameter of about 150 nm.

The mesoporous silica nanoparticles can have an average pore size of from about 1 nm to about 20 nm. For example, mesoporous silica nanoparticles can have an average pore size of from about 1 to about 20 nm in diameter. For example, the disclosed mesoporous silica nanoparticles can have an average pore size of from about 2 nm to about 20, from about 4 nm to about 20 nm, from about 6 nm to about 20 nm, from 8 to about 20 nm, from about 10 nm to about 20 nm, from about 3 nm to about 20 nm, from about 3 nm to about 12 nm, from about 3 nm to about 10 nm, from about 3 nm to about 8 nm, from about 3 nm to about 6 nm, from about 4 nm to about 12 nm, or from about 4 nm to about 10 nm. In still other examples, the mesoporous silica nanoparticles can have an average pore size of 1, 2, 4, 6, 8, 9, 10, 11, 12, 13, 14, 15, 16, 17, 18, 19, or 20 nm, where any of the stated values can form an upper or lower endpoint of a range. In specific examples, the mesoporous silica nanoparticles can have an average pore size of about 3, about 7, or about 11 nm.

The nanoparticles of gadolinium have an average diameter about the same size as the pores of the mesoporous silica nanoparticles. For example, the nanoparticles of gadolinium can have an average diameter of from about 1 nm to about 20 nm. In other examples, the nanoparticles of gadolinium can have an average diameter of from about 2 nm to about 20, from about 4 nm to about 20 nm, from about 6 nm to about 20 nm, from 8 to about 20 nm, from about 10 nm to about 20 nm, from about 3 nm to about 20 nm, from about 3 nm to about 12 nm, from about 3 nm to about 10 nm, from about 3 nm to about 8 nm, from about 3 nm to about 6 nm, from about 4 nm to about 12 nm, or from about 4 nm to about 10 nm. In still other examples, the disclosed nanoparticles of gadolinium can have an average diameter of 1, 2, 4, 6, 8, 9, 10, 11, 12, 13, 14, 15, 16, 17, 18, 19, or 20 nm, where any of the stated values can form an upper or lower endpoint of a range. In specific examples, the nanoparticles of gadolinium can have an average diameter of about 3, about 7, or about 11 nm.

The chelator can be 1,4,7-triazacyclononane-1,4,7-triacetic acid (NOTA), 1,4,7,10-tetraazacyclodode-cane-1, 4, 7,10-tetraacetic acid (DOTA), 1,4,8,11-tetraazacyclododenane-1,4,8,11-tetraacetic acid (TETA), 2,2′-(1,4,8,11-tetraazabicyclo[6.6.2]hexadecane-4,11-diyl)diacetic acid (CB-TE2A), 3,6,9,15-Tetraazabicyclo[9.3.1]pentadeca-1(15),11,13-triene-3,6,9-triacetic acid (PCTA), pendetide (GYK-DTPA), cyclohexyldiethylenetriaminepentaacetic acid (CHX-DTPA), 2-(4,7-biscarboxymethyl[1,4,7]triazacyclonona-1-yl-ethyl)carbonyl-methylamino]acetic acid (NETA), diethylene triamine pentaacetic acid (DTPA), desferrioxamine, nitrilotriacetate (NTA), DO3A, ethylenediammine, acetylacetonate, phenanthroline, oxalate, citric acid, bipyridine, cyanide, nitrite, acetonitrile, ethylenediamine tetraacetic acid (EDTA), ethylene glycol tetraacetic acid (EGTA), poly-1-lysine, polyethylenimine, or polyvinylpyrrolidone (PVP), or any salt, derivative, functionalized analog, or mixture of these. In a specific example, the chelator can be diethylenetriaminepentacetate.

The methods can also involve conjugating the gadolinium nanoparticles with a targeting agent, a dye molecule, a metal chelate, or a drug molecule. For example, one can use N-(3-(dimethylamino)propyl)-N′-ethylcarbodiimide hydrochloride (EDC) and N-hydroxysuccininmide (NHS) chemistry to conjugate peptides, like cyclic RGD peptides, onto the gadolinium nanoparticles. A wide variety of natural and synthetic molecules recognized by target cells can be used as the targeting moiety. Suitable targeting moieties include, but are not limited to, a receptor, ligand, polynucleotide, peptide, polynucleotide binding agent, antigen, antibody, or combinations thereof In one example, the targeting moiety is a peptide which has a length of from about 6 amino acids to about 25 amino acids.

### Compositions

Disclosed herein are nanoparticles where a metal is encapsulated in an amorphous carbon shell. The metal can be pure metal, metal oxide, metal complexes, or mixtures of these. These metal encapsulated carbon dots (herein referred to as MAC-dots) can have a wide variety of uses. In some specific examples, the metal is gadolinium; thus disclosed herein are Gd encapsulated carbon dots (hereafter referred to as Gd@C-dots). Also, reference to MAC-dots herein is meant to specifically include reference to Gd@C-dots.

Unlike most other nanocarriers/nanocapsules, carbon has low-toxicity and is highly biologically inert. Thus, the disclosed nanoparticle MAC-dots can remain intact even in harsh biological environments, therefore precluding the risk of metal release to the surroundings (Cao et al., Theranostics 2012, 2(3):295-301). With specific reference to gadolinium, stemming from the inert carbon coating, the disclosed nanoparticles are immune to the issue of Gd leaking that is often observed with complex-based Gd agents. Leakage of other metals from the disclosed nanoparticles is also expected.

The disclosed nanoparticles can have an average size of from about 1 to about 20 nm in diameter. For example, the disclosed nanoparticles can have an average size of from about 2 nm to about 20, from about 4 nm to about 20 nm, from about 6 nm to about 20 nm, from 8 to about 20 nm, from about 10 nm to about 20 nm, from about 3 nm to about 20 nm, from about 3 nm to about 12 nm, from about 3 nm to about 10 nm, from about 3 nm to about 8 nm, from about 3 nm to about 6 nm, from about 4 nm to about 12 nm, or from about 4 nm to about 10 nm. In still other examples, the disclosed nanoparticles can have an average size of 1, 2, 4, 6, 8, 9, 10, 11, 12, 13, 14, 15, 16, 17, 18, 19, or 20 nm nm, where any of the stated values can form an upper or lower endpoint of a range.

### Conjugates

The surface of the disclosed nanoparticles contains carboxyl groups that can be used to functionalize the surface of the nanoparticles. The carbonyl groups are electrophiles that can be used in nucleophilic substitution reactions or carbodiimide coupling reactions with any desirable functionalizing reagent. In certain examples, the functionalizing reagent can contain a targeting moiety that can be used to direct the functionalized nanoparticles to specific locations in the patient. Thus disclosed herein are MAC-dot nanoparticles functionalized with a targeting moiety. For example, RGD-peptides and cyclic RGD-peptides, when coupled to the disclosed MAC-dots, can direct the nanoparticles to target tumors. Similarly, EGFR targeting peptides, EGFR targeting therapeutics, VEGF targeting peptides, VEGF targeting therapeutics, and the like can be coupled/attached to the disclosed nanoparticles. Different types of antibodies, such as Herceptin, Avastin, and Erbitux, etc., can be coupled to the particle surface for facilitating particle targeting to tumors. Small molecule drugs such as doxorubicin, methotrexate or paclitaxel or their derivatives and can also be coupled to the surface of the nanoparticles, and in these cases the particles are used as drug carriers. Functionalizing the surface of the nanoparticles can also be used to assist the passage of the MAC-dots across certain cell membranes. For instance, the particle surface can be coated with a layer of positively charged polymer such as polyethylenimine and the resulting conjugates can be used as carriers for gene delivery (e.g., siRNA) due to assisting gene therapeutics passing through negatively charged cell membranes.

Suitable reagents for initiating a carbodiimide-mediate coupling to the carboxyl of the disclosed nanoparticles are commercially available. Specific examples of such reagents include, but are not limited to, water soluble carbodiimides such as 1-ethyl-3-(3-dimethylaminopropyl)-carbodiimide hydrochloride and 1-cyclohexyl-3-(2-morpholinoethyl)-carbodiimide-metho-p-toluene sulfonate, alcohol and water soluble N-ethyoxycarbonyl-2-ethoxy-1,2-dihydroquinoline, and organic soluble N,N′-dicyclohexylcarbodiimide.

### Formulations

While it can be possible for disclosed nanoparticles to be administered neat, it is also possible to present them as a pharmaceutical formulation. Accordingly, provided herein are pharmaceutical formulations which comprise one or more of the disclosed nanoparticles together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients can be used as suitable and as understood in the art; e.g., in Remington: The Science and Practice of Pharmacy, 21st Ed., Gennaro, Ed., Lippencott Williams & Wilkins (2003). The compositions and formulations disclosed herein can be manufactured in any manner known in the art, e.g., by means of conventional mixing, dissolving, granulating, dragee-making, levigating, emulsifying, encapsulating, entrapping or compression processes.

A nanoparticle as disclosed herein can be incorporated into a variety of formulations for therapeutic administration, including solid, semi-solid, or liquid forms. The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route can depend upon for example the condition and disorder of the recipient. The formulations can conveniently be presented in unit dosage form and can be prepared by any of the methods well known in the art of pharmacy. Typically, these methods include the step of bringing into association a compound or a pharmaceutically acceptable salt thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

The disclosed nanoparticles can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. The formulations can be presented in unit-dose or multi-dose containers, for example sealed ampoules and vials, and can be stored in powder form or in a freeze-dried (lyophilized) condition requiring only the addition of the sterile liquid carrier, for example, saline or sterile pyrogen-free water, immediately prior to use. Extemporaneous injection solutions and suspensions can be prepared from sterile powders, granules and tablets of the kind previously described.

Formulations for parenteral administration include aqueous and non-aqueous (oily) sterile injection solutions of the active compounds which can contain antioxidants, buffers, bacteriostats and solutes which render the formulation isotonic with the blood of the intended recipient; and aqueous and non-aqueous sterile suspensions which can include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the compounds to allow for the preparation of highly concentrated solutions.

### Methods of Using

The disclosed nanoparticles have properties that can afford them uses as optical, MRI, fluorescence, photoacoustic imaging probes. The disclosed nanoparticles can also be used in therapy (drug delivery, gene delivery, photodynamic therapy), catalysis, energy, and electronics applications.

In a specific example, the disclosed nanoparticles can be used a MRI/fluorescence dual modal imaging probes. For example, the disclosed nanoparticles, or formulations containing them, can be used as imaging agents to visualize cancerous tissues, e.g., tumors. In one aspect, disclosed herein is a method of detecting cancer in vivo comprising administering a nanoparticle (e.g., Gd@C-dot) as disclosed herein to an individual and detecting a fluorescent signal and/or magnetic resonance signal. Also, a region of interest in the individual can be imaged using a fluorescence reflectance imaging system (such as the F-Pro from Bruker), which is fitted with multiple band pass filters for excitation and emission.

In recent years, many new fluorescence imaging systems, such as endoscopes (Hsiung et al., Nat Med 14:454 (2008); Funovics et al., Mol Imaging 2:350 (2003)), wide-field video cameras (Knapp et al., European urology 52:1700 (2007); van Dam et al., Nat Med 17:1315 (2011)), and goggles (Liu et al., Surgery 149:689 (2011); Wang et al., J Biomed Opt 15:020509 (2010)), have been developed. Any of these systems can be used to detect the fluorescent signal, or lack thereof, in an individual to whom the disclosed nanoparticles have been administered. Further, the development of the fluorescence can be followed using a near infrared video camera (e.g., Fluoptics).

The disclosed nanoparticles can also be used as MR imaging probes. The nanoparticles disclosed herein can be used to detect/image a variety of other cancers. Examples of cancer types detectable by the compounds and compositions disclosed herein include bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer. Further examples include cancer and/or tumors of the anus, bile duct, bone, bone marrow, bowel (including colon and rectum), eye, gall bladder, kidney, mouth, larynx, esophagus, stomach, testis, cervix, mesothelioma, neuroendocrine, penis, skin, spinal cord, thyroid, vagina, vulva, uterus, liver, muscle, blood cells (including lymphocytes and other immune system cells). Specific cancers contemplated for imaging include carcinomas, Karposi's sarcoma, melanoma, mesothelioma, soft tissue sarcoma, pancreatic cancer, lung cancer, leukemia (acute lymphoblastic, acute myeloid, chronic lymphocytic, chronic myeloid, and other), and lymphoma (Hodgkin's and non-Hodgkin's), and multiple myeloma.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Transmission electron microscopy (TEM) was carried out on a FEI Tecnai20 transmission electron microscope operating at 200 kV accelerating voltage. Fluorescence spectra were recorded on a Hitachi F-7000 fluorescence spectrophotometer. UV-Vis absorbance spectra were obtained on a BioTek Synergy MX multi-mode microplate reader. Fluorescence QY was measured using quinine sulfate in 0.1 M H₂SO₄ (literature quantum yield: 58% at 354 nm excitation) as a reference standard (Bhunia, S., et al., Sci. Rep. 2013, 3:1473). Zeta potential measurements were carried out on a Malvern Zetasizer Nano ZS system. Fourier transform infrared (FT-IR) spectra were recorded on a Nicolet iS10 FT-IR Spectrometer. TGA was performed on a Mettler TGA/SDTA851 system and the data was analyzed by STAR software, version 8.10.

Animal studies were performed according to a protocol approved by the Institutional Animal Care and Use Committee (IACUC) of University of Georgia. The U87MG tumor models were generated by subcutaneously injecting 5×10⁶ cells in 100 μL PBS into the right hindlimb of 4-6 week athymic nude mice (Harlan).

Quantitative data were expressed as mean ±s.e.m.

### Example 1: Synthesis of MSNs of Different Pore Sizes

MSNs were prepared following a published procedure through co-coagulation of tetraethyl orthosilicate (TEOS) and [3-(2-Aminoethylamino)propyl]trimethoxysilane (APS) (volume ratio between TEOS and APS was 1/0.6) using CTAB as the template (Chen, H., et al., Theranostics 2013, 3:650). Briefly, 0.6 g of cetyltrimethylammonium bromide (CTAB) was dissolved in 300 mL water. With magnetic stirring, 2.1 mL of 2 M NaOH was added, and the resulting solution was heated up to 70° C. Subsequently, 3 mL TEOS, 18 mL ethylacetate, and 1.8 mL of APS were added, and the mixture was stirred at 70° C. for 3 h. The raw products were collected by centrifugation, washed 3 times with ethanol, and re-dispersed in ethanol. To remove CTAB, 50 mg of NH₄NO₃ was added to the particle suspension and the solution was stirred for 3 h at 60° C. The as-synthesized silica nanoparticles had an average diameter of 160 nm and a pore size of ˜3 nm (FIG. 1B). The resulting particles (MSN-3) were washed twice with ethanol, and dried at 60° C. overnight. To prepare MSN-7 and MSN-11, 5 mg MSN-3 was incubated in a Na₂CO₃ solution (pH 12) at 50° C. for 30 min and 60 min, respectively (Chen, Y., et al., Small 2011, 7:2935; Chen, Y., et al., ACS Nano 2010, 4:529) to be further rendered porous (Huang, Y., et al., Mol. Pharmaceut. 2014, 11:3386). MSNs with average pore sizes of 7 and 11 nm, were obtained (FIG. 1B).

The resulting MSN-3, MSN-7, and MSN-11 were analyzed by Fourier transform infrared spectroscopy (FT-IR). For all of the three MSN formulations, three major peaks at ˜1400, ˜1100, and ˜700 cm⁻¹were observed. These were attributable to the absorbance by C—N, Si—O, and Si—C stretches, respectively (FIG. 1C). MSN-3 showed positive surface charge in water (zeta potential of 40.2 mV, FIG. 8A), which is attributed to the surface primary and secondary amine groups inherited from APS (Soto-Cantu, E., et al., Langmuir 2012, 28:5562; Graf, C., et al., Langmuir 2012, 28:7598). Compared to MSN-3, MSN-7 and MSN-11 manifested similar surface charge (zeta potentials of 39.3 and 36.8 mV for MSN-7 and MSN-11, respectively, FIG. 8A). This indicates that the alkaline etching mostly was occurred to the interiors and minimally affected the surface (Huang, Y., et al., Mol. Pharmaceut. 2014, 11:3386). Thermogravimetric analysis (TGA) found a weight drop of ˜33% between 150° C. and 650° C., which was attributable to the oxidization of the amine groups (FIG. 1D). Compared to MSN-3, MSN-7 and MSN-11 manifested a similar level of surface charge (zeta potential 39.3 and 36.8 mV, for MSN-7 and MSN-11, respectively). This indicates that the alkaline etching occurred mostly to the interiors of the particles and had little impact to the surface (Id.).

### Example 2: Synthesis of Gd@C-Dots

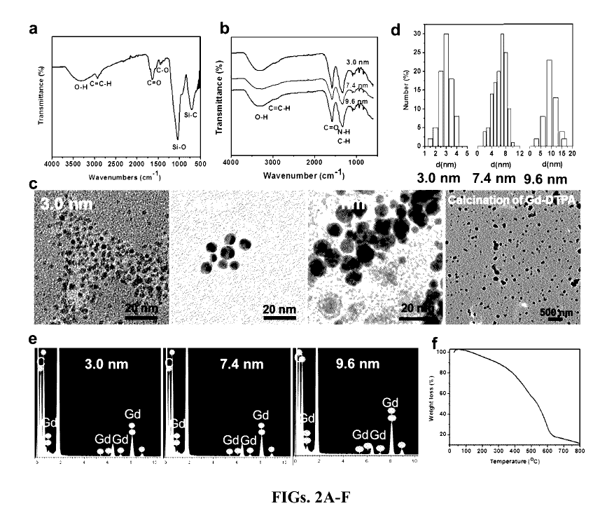

Gd(NO₃)₃ and Gd-DTPA were mixed with MSNs of different pore sizes. Specifically, MSN-3, -7, and -11 were incubated in a solution containing 100 mM Gd(NO₃)₃ and 10 mM Gd-DTPA (FIG. 1A). After purification by centrifugation, the Gd-loaded particles were calcined in a muffle furnace at 200° C. for 2 h in the open air. FT-IR analysis found that after calcination, the N—C peak (˜1400 cm⁻¹) disappeared, suggesting that amines in the silica matrix were oxidized during the process (FIG. 2A). This corroborates with zeta potential analyses, which found a slightly negative surface charge after calcination (−1.29 mV, FIG. 8B).

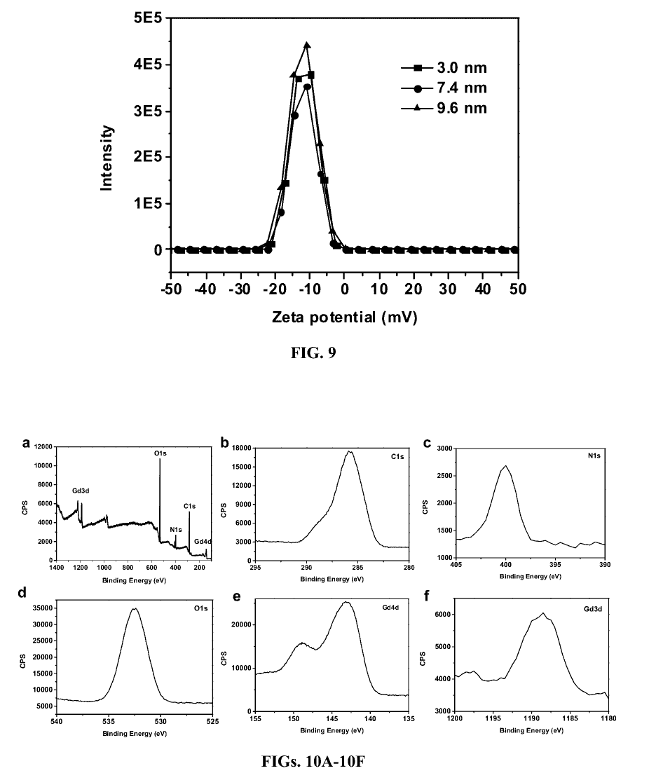

The resulting nanoparticles were incubated in NaOH (6 M) for 12 h to melt down the silica framework. The final products were obtained by dialysis against or an ultracentrifugal unit (MWCO=10K). FT-IR analyses found no Si—O and Si—C peaks (˜1100 and ˜700 cm⁻¹, respectively) with the purified products, indicating complete removal of the silica contents (FIG. 2B) (Wang, F., et al., Adv. Funct. Mater. 2011, 21:1027). Meantime, characteristic absorbance of C═C—H and —C═O stretches (˜2900 and ˜1600 cm⁻¹, respectively) were observed (FIG. 2B), confirming the formation of carbon nanoparticles. Moreover, broad absorbance around 3300 cm⁻¹ was also observed, suggesting the presence of multiple carboxyl groups on the nanoparticle surface. This corroborated well with the zeta potential analysis, which found a negatively charged surface (−12.2 mV, FIG. 9).

The TEM analysis showed that the resulting Gd@C-dots were homogenous in size (FIGS. 2C and 2D) (Chen, H., et al., Adv. Mater. 2014, 26:6761). Specifically, the average sizes were 3.0±0.5, 7.4±1.2, and 9.6±2.0 nm for Gd@C-dots made from MSN-3, -7 and -11, respectively (FIGS. 2C and 2D). These sizes match well with the pore sizes of the corresponding MSNs. As a comparison, raw products made from direct calcination of Gd complexes contained carbon species of a broad range of sizes and structures (FIG. 2B, FIGS. 8A and 8B) (Id.).

Energy-dispersive X-ray spectroscopy (EDX) confirmed the presence of Gd in the nanoparticles. Taking 3 nm Gd@C-dots for instance, Gd accounted for 5.2% of the total mass (FIG. 2E); the other two major elements were carbon (53.73%) and oxygen (31.50%), respectively. The results corroborate well with the X-ray photoelectron spectroscopy (XPS) analysis, which found that the carbon, oxygen and gadolinium fractions were 56.4%, 38.7%, and 4.9%, respectively (FIGS. 10A-10F). Moreover, XPS detected absorbance of C1s and O1s in Gd@C-dots, suggesting the presence of hydroxyl and carboxyl groups on the nanoparticle surface (FIGS. 10A-10F). Meanwhile, TGA found a weight loss of ˜75% between 100 and 600° C., which was mainly attributed to the oxidization of carbon (FIG. 2F).

Optical Properties of Gd@C-Dots:

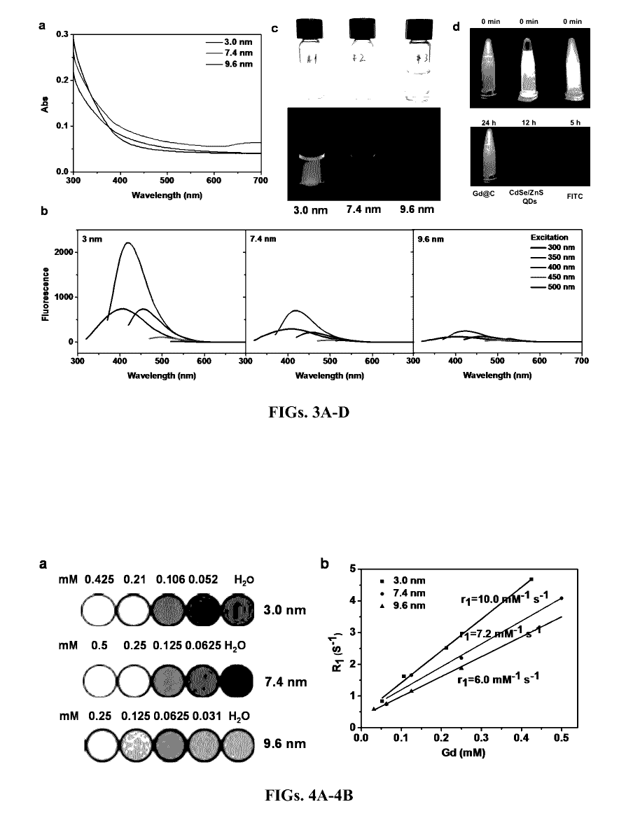

All of the Gd@C-dot formulations showed broad absorbance in the visible region (FIG. 3A). They were also all highly fluorescent, excited by visible light of a wide range of wavelengths to emit strong photoluminescence (FIG. 3B). The intensity of the luminescence, however, is largely dependent on the nanoparticle size, with the strongest fluorescence observed in 3.0 nm Gd@C-dots and the weakest with 9.6 nm Gd@C-dots (FIGS. 3B and 3C). To quantitatively assess the fluorescence, we measured quantum yields (QYs) of the three Gd@C-dot formulations using quinine sulfate as a reference (excitation at 360 nm) (Bhunia, S., et al., Sci. Rep. 2013, 3:1473). It was determined that the QYs were 30.2%, 12.3% and 1.6%, for the 3.0, 7.4 and 9.6 nm Gd@C-dots, respectively. The fluorescence of Gd@C-dots was highly resistant against photo-bleaching. FIG. 3D shows a comparison among Gd@C-dots, fluorescein, and CdSe/ZnS quantum dots (QDs). Under continuous UV irradiation, fluorescein was completely bleached within minutes and CdSe/ZnS QDs was bleached within 12 h. For Gd@C-dots, on the other hand, there was no drop of luminescence intensity despite of irradiation for over 24 h.

MRI Phantom Studies.

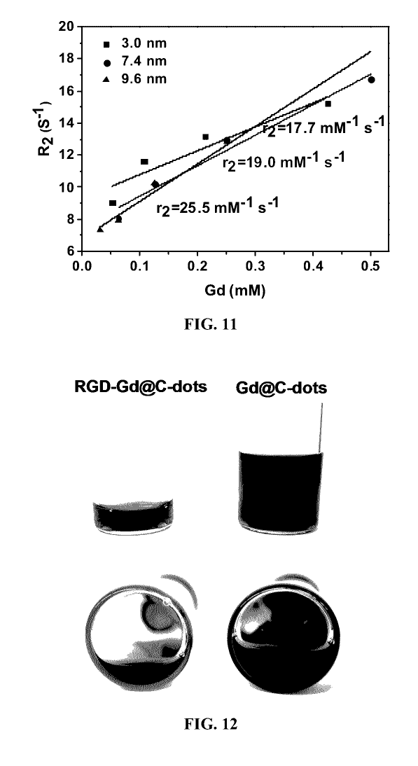

The contrast ability of the Gd@C-dots was evaluated by MRI phantom studies. Gd@C-dots of elevated concentrations (0-0.5 mM) were suspended in 1% agarose gel in 300 μl PCR tubes. These tubes were then embedded in a home-made tank designed to fit the MRI coil. T₁-weighted MR images of the samples were acquired on a 7T Varian small animal MRI system using the following parameters: TR/TE=500/12 ms (T₁), 256×256 matrices, and repetition times=4. The three Gd@C-dots showed comparable and relatively low r₂ relaxivities, which were 17.7, 19.0, and 25.5 mM⁻¹s⁻¹, respectively, for 3.0, 7.4, and 9.6 nm Gd@C-dots (FIG. 11). Meanwhile, the r₁ relaxivities were found to be inversely correlated to the particle size (FIG. 4A). Specifically, r₁ values were 10.0, 7.2, and 6.0 mM⁻¹s⁻¹ for 3.0, 7.4, and 9.6 nm Gd@C-dots, respectively (FIG. 4B). As a comparison, r₁ for Gd-DTPA is 3.1 mM⁻¹s⁻¹ at 7T (Kim, T., et al., J. Am. Chem. Soc. 2011, 133:2955; Kalavagunta, C., et al., Contrast media Mol. Imaging 2014, 9:169). By convention, compounds with r₂/r₁ ratios of less than 5 are considered primarily T₁ contrast agents, whereas those larger than 10 are considered T2 contrast agents (Caravan, P., et al., Chem. Rev. 1999, 99:2293). Hence, all three formulations are good T₁ contrast agents (r₂/r₁ ratios of 1.77, 2.64, and 4.25 for 3.0, 7.4 and 9.6 nm Gd@C-dots, respectively). Due to the most prominent magnetic and optical properties, 3.0 nm Gd@C-dots were chosen for the subsequent in vitro and in vivo studies.

Physical Stablity and Photo-Stability of Gd@C-Dots.

Gd@C-dots were incubated in solvents of different pH (4-7.4) at 37° C., and the fluorescence intensity over time was monitored (ex/em: 360/425 nm). While pH 7.4 is close to the physiological pH, pH 4.0 is close to or lower than the pH in cell endosomes/lysosomes (Casey, J., et al., Nature Rev. Molec. Cell Bio. 2010, 11:50). In all of the tested solutions, there was no change of luminescence intensity despite of long-time incubation (FIG. 4A).

The amount of Gd(III) released from Gd@C-dots was measured by using xylenol orange as a Gd marker (Barge, A., et al., Cont. Media Mol. 12006, 1:184; Caravan, P., et al., Angew. Chem. Int. Ed. 2007, 46:8171; Abada, S., et al., Chem. Comm. 2012, 48:4085). At both pH, zero Gd leakage was observed, confirming the great physiological stability of the nanoparticles in that Gd was well encased within carbon and not liberated in an acidic environment (FIG. 4B).

For photo-stability, 3.0 nm Gd@C-dots, fluorescein, and CdSe@ZnS QDs were irradiated continuously by a UV lamp (254 nm, 30W), and their fluorescence was monitored. Gd@C-dots are stable in aqueous solutions. They can be kept for over 6 months without visible precipitation (FIG. 12). The extraordinary colloidal stability is believed to be attributed to not only the negatively charged surface but also the low density of the nanoparticles, given that the major component is carbon.

Cytotoxicity:

The cytotoxicity of Gd@C-dots was evaluated with U87MG (human glioma) cells using 3-(4, 5-dimethylthiazolyl-2)-2,5-diphenyltetrazolium bromide (MTT) assays. 2.5 mM Ca(II) was added into the incubation medium; while posing no direct toxicities to cells, such a high concentration of calcium can cause transmetallation to Gd complexes, leading to liberation of free Gd (Corot, C., et al., J. Magn. Reson. Imaging 1998, 8:695). Taking Gd-DTPA for instance, while showing no toxicities under normal cell culture conditions, the compound became highly toxic in the presence of 2.5 nM Ca(II) (IC₅₀ of 33.1 μg/mL, FIG. 5C) (Wu, X., et al., Pharm. Res. 2010, 27:1390; Aime, S., et al., J. Magn. Reson. Imaging 2009, 30:1259). As a comparison, Gd@C-dots caused neither cell viability drop nor noticeable cell morphology change even at 100 μg/mL (FIGS. 5C and 5D). The low toxicity was again attributed to the great resistance of Gd@C-dots against biodegradation and transmetallation.

### Example 3: Gd@C-Dot Conjugation with c(RGD)yK

Given that there are multiple carboxyl groups on the surface of Gd@C-dots, the particles can be easily conjugated with functional molecules. In this example, c(RGDyK), a tumor targeting ligand, was coupled to the particle surface using N-(3-(dimethylamino)propyl)-N′-ethylcarbodiimide hydrochloride (EDC) and N-hydroxysuccininmide (NHS) chemistry (Xing, Y., et al., Nat. Protoc. 2007, 2:1152). Specifically, Gd@C-dots were dispersed in a borate buffer (pH 8.3). Into the solution, carbodiimide (EDC) and N-hydroxysuccinimide (NHS) (100×) in DMSO was added, and the mixture was magnetically stirred for 30 min. The intermediate was purified by centrifugation, and redispersed in PBS (pH 7.4). Into the solution, c(RGDyK) in DMSO (20×) was added and the mixture was incubated for 2 h with gentle agitation. The product was collected using a centrifugal filtration unit (Millipore filter unit: MWCO 3K) and redispersed in PBS (pH=7.4).

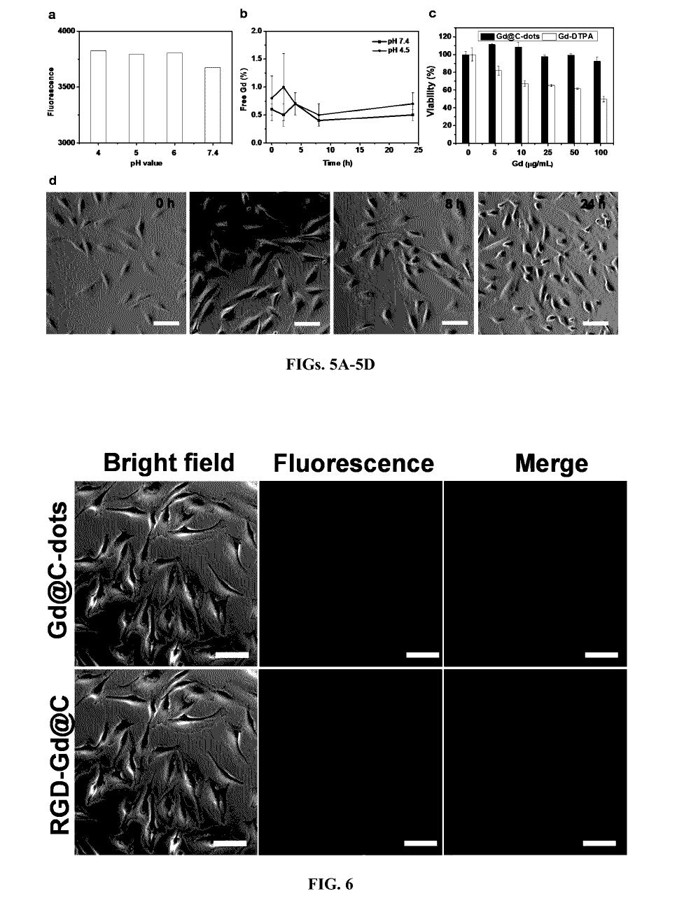

c(RGDyK) affords strong binding affinity toward integrin αᵥβ₃, a cancer biomarker that is seen overexpressed on neoplastic blood vessels and in many types of cancer cells. So the resulting conjugates (hereafter designated as RGD-Gd@C-dots) and unmodified Gd@C-dots were incubated with U87MG cells and imaged under a fluorescence microscope (ex/em: 360/460 nm). Specifically, U87MG cells were cultured in DMEM supplemented with 2 mM L-glutamine, 1.5 g/L sodium bicarbonate, 0.1 mM nonessential amino acids, 1.0 mM sodium pyruvate, and 10% fetal bovine serum at 37° C. in a humidified atmosphere with 5% CO₂. 10⁵ U87MG cells were seeded in 96-well plates (1×10⁴ cells per well) 24 h prior to the experiments. RGD-Gd@C-dots and Gd@C-dots at different concentrations were added to the medium and incubated with the cells for 24 h. MTT assays were then performed. The incubation was stopped after 1 h, and the cells were rinsed 3 times with PBS (pH 7.4). The slides were mounted and imaged under an Olympus X71 fluorescence microscope. While Gd@C-dots showed little cell uptake, strong fluorescence was observed in the cytoplasm of cells incubated with RGD-Gd@C-dots, suggesting RGD-integrin mediated cell internalization (FIG. 6).

### Example 4: In vivo MRI

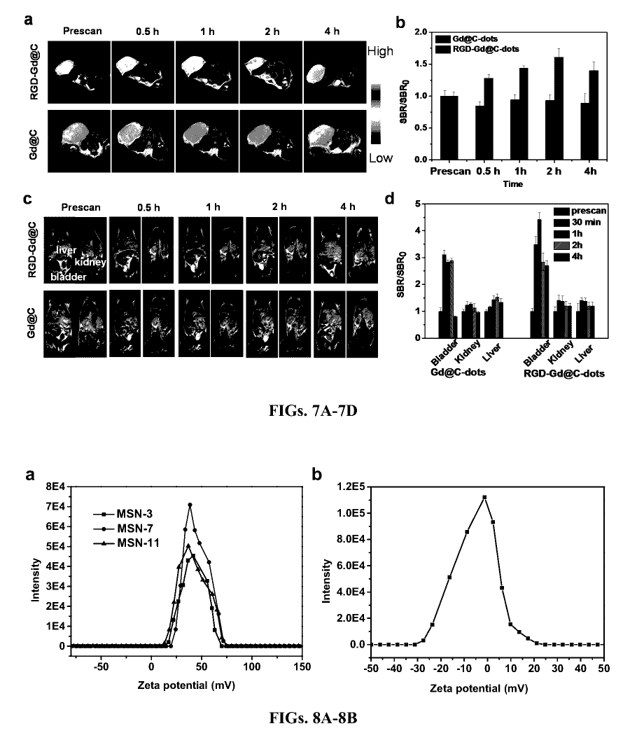

The capacity of RGD-Gd@C-dots as a tumor targeting probe was evaluated in U87MG subcutaneous tumor models. The imaging studies were performed in U87MG tumor-bearing mice when the tumors reached a size of ˜250 mm³. RGD-Gd@C-dots and Gd@C-dots at the same amount (0.1 mmol Gd/kg) were intravenously injected (n=3). For controls, Gd@C-dots were injected at the same Gd dose. Transverse and coronal T₁-weighted MR images were acquired at 0, 30 min, 60 min, 2 h, and 4 h post the nanoparticle injection using the following parameters: TR/TE=500/12 ms, field-of-view (FOV)=70×70 mm², matrix size=256×256, slice=4, and thickness=1 mm (FIGS. 7A-D). To quantify the signal change, we calculated the signal-to-background ratio (SBR) by finely analyzing regions of interest (ROIs) of the MR images and calculated the values of SBR/SBR₀ to represent the signal changes (Huang, J., et al., ACS Nano 2010, 4, 7151; Zhou, Z., et al., ACS Nano 2013, 7:3287). Signal intensity (SI) of normal live, kidney, bladder, muscle, and tumor were measured before and after injection of Gd@C nanoparticles. The mean SI measurements of 3 mice per group were used for statistical analysis. Because of slight changes in the position of the mice at different imaging stages, pre and post ROIs were determined manually on each image as reproducible as possible. For each animal, 3-5 ROIs were selected to measure the SI of the liver, kidney, bladder, muscle, and tumor. The SBR values were calculated according to SBR=SI_{organ}/SI_{muscle}.

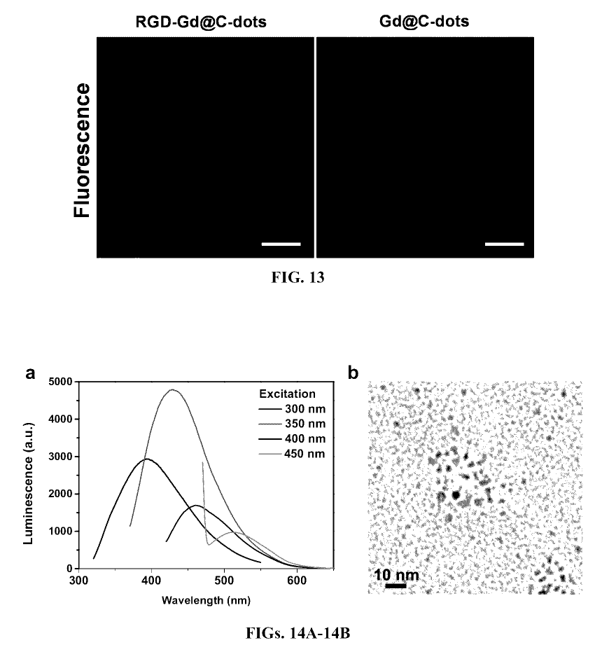

For RGD-Gd@C-dots, region of interest (ROI) analyses found clear signal enhancement in tumors, which was peaked at 2 h (relative signal enhancement, i.e. SBR/SBR₀, is 61.3±13.9%. For Gd@C-dots, the signal enhancement was not significant at all of the time points (FIGS. 7A and 7B; FIG. 13).

Signal changes in normal tissues were also examined. For RGD-Gd@C-dots, there was a certain degree of signal increase in the liver and kidneys at early time points (FIGS. 7C and 7D). Specifically, relative to the pre-scans, the signals in the liver and kidneys at 1 h were increased by 44±4% and 28±13%, respectively. The signals then gradually decayed (after 4 h SBR/SBR₀=1.34±0.15 and 0.97±0.030 for the liver and kidneys, respectively). For the kidneys in particular, the signals dropped to the normal level after 4 h (SBR/SBR₀=0.97±0.03).

Meantime, strong signals were observed in the bladder shortly after the Gd@C-dots injection (FIGS. 7C and 7D), indicating efficient renal clearance of the nanoparticles. To further investigate, we collected urine samples from the animals ˜120 min after the injection and harvested the nanoparticles in the urine by centrifugation. TEM analysis confirmed the presence of a large amount of intact Gd@C-dots (FIG. 14A). This was corroborated by the detection of strong photoluminescence that was characteristic of Gd@C-dots (FIG. 14B). Overall, the results confirmed that RGD-Gd@C-dots can selectively home to tumors, with the unbound particles efficiently excreted through renal clearance, which is ideal for imaging.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of forming nanoparticles of gadolinium encapsulated in an amorphous carbon shell, comprising:
calcining a mixture comprising a mesoporous silica nanoparticle, a gadolinium-containing compound, and a chelator, thereby forming the nanoparticles of gadolinium within the mesoporous silica nanoparticle; and
removing the mesoporous silica nanoparticle from the nanoparticles of gadolinium.

2. The method of claim 1, wherein the mesoporous silica nanoparticle is removed by dissolving it in a base and isolating the nanoparticles of gadolinium.

3. The method of claim 2, wherein the base is sodium hydroxide.

4. The method of claim 1, wherein the mesoporous silica nanoparticle has an average diameter of from about 100 nm to about 200 nm.

5. The method of claim 1, wherein the mesoporous silica nanoparticle has an average pore size of from about 1 nm to about 20 nm.

6. The method of claim 1, wherein the mesoporous silica nanoparticle has an average pore size of about 3, about 7, or about 11 nm.

7. The method of claim 1, wherein the mesoporous silica nanoparticle is prepared by contacting a tetraalkyl orthosilicate and organofunctionalized silane in the presence of a tetraalkylammonium halide.

8. The method of claim 7, wherein the tetraalkyl orthosilicate is tetraethylorthosilicate.

9. The method of claim 7, wherein the organofunctionalized silane is [3-(2-Aminoethylamino)propyl]trimethoxysilane.

10. The method of claim 7, wherein the tetraalkylammonium halide is cetyltrimethylammonium bromide.

11. The method of claim 1, wherein the nanoparticles of gadolinium have an average diameter of from about 1 nm to about 20 nm.

12. The method of claim 1, wherein the chelator is diethylenetriaminepentacetate.

13. The method of claim 1, wherein the chelator is 1,4,7-triazacyclononane-1,4,7-triacetic acid (NOTA), 1,4,7,10-tetraazacyclodode-cane-1, 4, 7,10-tetraacetic acid (DOTA), 1,4,8,11-tetraazacyclododenane-1,4,8,11-tetraacetic acid (TETA), 2,2′-(1,4,8,11-tetraazabicyclo[6.6.2]hexadecane-4,11-diyl)diacetic acid (CB-TE2A), 3,6,9,15-Tetraazabicyclo[9.3.1]pentadeca-1(15),11,13-triene-3,6,9-triacetic acid (PCTA), pendetide (GYK-DTPA), cyclohexyldiethylenetriaminepentaacetic acid (CHX-DTPA), 2-(4,7-biscarboxymethyl[1,4,7]triazacyclonona-1-yl-ethyl)carbonyl-methylamino]acetic acid (NETA), diethylene triamine pentaacetic acid (DTPA), desferrioxamine, nitrilotriacetate (NTA), DO3A, ethylenediammine, acetylacetonate, phenanthroline, oxalate, citric acid, bipyridine, cyanide, nitrite, acetonitrile, ethylenediamine tetraacetic acid (EDTA), ethylene glycol tetraacetic acid (EGTA), poly-1-lysine, polyethylenimine, or polyvinylpyrrolidone (PVP), or any salt, derivative, functionalized analog, or mixture of these.

14. A composition, comprising: nanoparticles of gadolinium encapsulated in an amorphous shell and a mesoporous silica nanoparticle.

15. The composition of claim 14, wherein the mesoporous silica nanoparticle has an average diameter of from about 100 nm to about 200 nm.

16. The composition of claim 14, wherein the mesoporous silica nanoparticle has an average pore size of from about 1 nm to about 20 nm.

17. The composition of claim 14, wherein the mesoporous silica nanoparticle has an average pore size of about 3, about 7, or about 11 nm.

18. The composition of claim 14, wherein the nanoparticles of gadolinium have an average diameter of from about 1 nm to about 20 nm.

19. The composition of claim 14, wherein the nanoparticles of gadolinium are conjugated to a targeting moiety.

20. The composition of claim 19, wherein the targeting moiety is a cyclic RDG peptide.
